(19) United States
(12) Patent Application Publication
(10) Pub. No.: US 2024/0281968 A1
(43) Pub. Date: Aug. 22, 2024

(54) SYSTEM AND METHOD FOR RETINAL OPTICAL COHERENCE TOMOGRAPHY CLASSIFICATION USING REGION-OF-INTEREST AWARE RESNET

(71) Applicant: MG Health Tech LLC, Allen, TX (US)

(72) Inventors: Manikandan Muthu, Salem (IN); Ganesh Natesan, Edaipadi (IN); Dhurika V, TN (IN)

(21) Appl. No.: 18/648,374

(22) Filed: Apr. 27, 2024

(51) Int. Cl. G06T 7/00; A61B 3/10; A61B 3/12; G06T 5/30; G06T 7/13; G06V 10/44; G06V 10/764; G06V 10/82

(52) U.S. Cl. CPC G06T 7/0012; A61B 3/102; A61B 3/1225; G06T 5/30; G06T 7/13; G06V 10/44; G06V 10/764; G06V 10/82; G06T 2200/04; G06T 2207/10101; G06T 2207/20084; G06T 2207/30041; G06V 2201/03

(57) ABSTRACT

A retinal optical coherence tomography (OCT) image analysis (ROCTIA) system (1200) and method for analyzing one or more retinal scan images of an eye of a user to identify one or more retinal conditions of the eye. The system includes a scanner device (1202) and a processor (1204) configured with a Region-of-Interest Aware (ROI-Aware) Residual Network (ResNet). The scanner device (1202) configured to scan the eye of the user to obtain the one or more retinal scan images of the eye of the user. The processor (1204) classifies each of the one or more retinal scan images based on a region of interest (ROI) in each of the one or more retinal scan images. The ROI is obtained in real-time while the one or more retinal scan images are obtained. The processor (1204) identifies one or more retinal conditions of the eye based on one or more retinal scan images.

### TECHNICAL FIELD

The present invention is directed to diagnosis of eye diseases and more particularly to system and method for optical coherence tomography image classification using region-of-interest aware RESNET.

### BACKGROUND

Retinal diseases and disorders pose significant challenges to the healthcare industry, demanding precise and timely diagnostic solutions. The field of medical imaging, specifically retinal optical coherence tomography (OCT), has witnessed significant advancements in recent years. Optical Coherence Tomography (OCT) has emerged as a powerful imaging technology for capturing detailed cross-sectional views of the retina, aiding in the diagnosis of conditions such as age-related macular degeneration, diabetic retinopathy, and glaucoma. Retinal optical coherence tomography (OCT) technique (interchangeably termed only as optical coherence tomography or OCT imaging or just OCT), has witnessed significant advancements in recent years. OCT imaging plays a pivotal role in the diagnosis and management of various eye conditions. It provides high resolution cross-sectional images of the retinal layers, allowing ophthalmologists and optometrist to access the health of the eye.

Despite the advancements in OCT imaging, the accurate classification of retinal scans remains intricate. Further, accurate classification of images generated by OCT imaging (OCT scans) and diagnosis thereupon remains a complex task. This is because, despite its diagnostic capabilities, retinal OCT image analysis presents a set of intricate challenges as set out under.

One challenge is Region of Interest (ROI) Identification. Within these scans, certain regions contain crucial diagnostic information. Identifying these ROIs within the vast data is a complex task and can vary from patient to patient and condition to condition.

Another challenge is accuracy and consistency. Ensuring consistent and accurate diagnoses is a challenge, as the manual interpretation of OCT scans depends on the expertise of the clinician. Variability in assessments can lead to misdiagnoses or missed early signs of diseases.

Yet another challenge is timeliness of diagnosis. Early detection is vital in many eye conditions, as prompt intervention can prevent irreversible vision loss. The time taken for manual analysis can hinder timely diagnosis and treatment.

Hence there is a need in the art for a system and method to advance the field of ophthalmic healthcare by automating and enhancing the analysis of retinal OCT scans that lessens or eliminates above mentioned challenges.

### SUMMARY

The present invention is directed to diagnosis of eye diseases and more particularly to system and method for optical coherence tomography image classification using region-of-interest aware RESNET.

The invention offers a comprehensive solution to problems in the field of retinal Optical Coherence Tomography by introducing a “ROI-Aware 2D ResNet” (Region of Interest-Aware 2D Residual Neural Network) for retinal OCT classification. This novel deep learning model is specifically designed for analyzing retinal scans, with a keen focus on improving efficiency and accuracy.

It is an object of the present disclosure provide for system and method for optical coherence tomography image classification using region-of-interest aware residual networks (RESNET) that automates and enhances the analysis of retinal OCT scans.

It is another object of the present disclosure to provide for a system that provides consistently accurate and reliable diagnoses by automating the identification of relevant regions within OCT scans.

It is yet another object of the present disclosure provides for a system to expedite the diagnostic process, enabling timely treatment for eye conditions and reducing the workload on medical professionals.

It is an object of the present disclosure provide for a system that makes expert-level retinal OCT analysis more accessible, bridging the gap in regions with limited ophthalmic expertise.

It is an object of the present disclosure provide for a system that is scalable and can address the increasing volume of retinal OCT scans with a systematic and efficient approach, ensuring high-quality eye care services

The key features of the invention can be summarized as follows:

Efficient Processing: The ROI-Aware 2D ResNet streamlines the processing of retinal scans. By focusing on the region of interest within each scan, it eliminates the need to analyze irrelevant or redundant data, significantly reducing processing time.

Accuracy: The model's architecture is designed to enhance the accuracy of diagnostic results. It leverages the power of deep learning to detect subtle abnormalities or patterns indicative of various eye conditions, ensuring a more precise diagnosis

Scalability: The system is built to adapt to the increasing volume of retinal scans. As the number of patients seeking retinal OCT scans rises, the invention can efficiently accommodate this growth, ensuring that diagnostic services remain of high quality.

Timely Diagnoses: By improving efficiency, the invention ensures that patients receive their diagnoses promptly. Swift identification of eye conditions is critical for early intervention and preventing the progression of diseases, ultimately leading to better patient outcomes.

Streamlined Workflow: The invention integrates seamlessly into the existing workflow of eye care providers, reducing the burden on ophthalmologists and healthcare staff. It simplifies the process of scan analysis and reporting.

### BRIEF DESCRIPTION OF THE DRAWINGS

The diagrams are for illustration only, which thus is not a limitation of the present disclosure, and wherein:

FIG. 1 illustrates brief steps for region of interest (ROI) extraction, in accordance with an exemplary embodiment of the present disclosure.

FIG. 2 illustrates an image obtained upon performing image enhancement through histogram equalization, improving contrast, and revealing hidden details, as one of the ROI extraction steps, in accordance with an exemplary embodiment of the present disclosure.

FIG. 3 illustrates an image obtained upon performing binary conversion: enhanced image transformed into a binary representation for further analysis, as one of the ROI extraction steps, in accordance with an exemplary embodiment of the present disclosure.

FIG. 4 illustrates an image obtained upon performing inverted binary image, as one of the ROI extraction steps, in accordance with an exemplary embodiment of the present disclosure. This transformation reverses the values of a binary image, creating a negative rendition of the critical areas

FIG. 5 illustrates an image obtained upon performing canny edge detection, as one of the ROI extraction steps, in accordance with an exemplary embodiment of the present disclosure. The Canny edge detection algorithm is then employed to identify edges and transitions within the image

FIG. 6 illustrates an image obtained upon performing morphological operations, as one of the ROI extraction steps, in accordance with an exemplary embodiment of the present disclosure. The image is subjected to morphological operations, including dilation and erosion, to enhance specific features and remove noise

FIG. 7 illustrates an image obtained upon performing ROI mask, as one of the ROI extraction steps, in accordance with an exemplary embodiment of the present disclosure. This mask defines the Region of Interest (ROI), non-essential areas.

FIG. 8 illustrates an image obtained upon performing cleared region removed from ROI, as one of the ROI extraction steps, in accordance with an exemplary embodiment of the present disclosure. Illustrating the removal of undesired areas from the Region of Interest (ROI).

FIG. 9 illustrates an image obtained upon performing difference image, as one of the ROI extraction steps, in accordance with an exemplary embodiment of the present disclosure. This image captures the contrast between the extracted Region of Interest (ROI) and the surrounding unwanted portions.

FIG. 10 illustrates an exemplary architecture of ResNet-18 (2D ResNet/3D ResNet) as well know in the art. For example in https://doi.org/10.1371/journal.pone.0256630.g005.

FIG. 11 elaborates upon a computer implemented method of performing classification of an image obtained using Optical Coherence Tomography (OCT), in accordance with an exemplary embodiment of the present disclosure.

FIG. 12 relates to a retinal optical coherence tomography (OCT) image analysis (ROCTIA) system for analyzing one or more retinal scan images of an eye of a user to identify one or more retinal conditions of the eye, in accordance with an exemplary embodiment of the present disclosure.

FIG. 13 relates to a method for analyzing one or more retinal scan images of an eye of a user to identify one or more retinal conditions of the eye, in accordance with an exemplary embodiment of the present disclosure.

### DETAILED DESCRIPTION

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and firmware and/or by human operators.

Embodiments of the present invention may be provided as a computer program product, which may include a machine-readable storage medium tangibly embodying thereon instructions, which may be used to program a computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

Various methods described herein may be practiced by combining one or more machine-readable storage media containing the code according to the present invention with appropriate standard computer hardware to execute the code contained therein. An apparatus for practicing various embodiments of the present invention may involve one or more computers (or one or more processors within a single computer) and storage systems containing or having network access to computer program(s) coded in accordance with various methods described herein, and the method steps of the invention could be accomplished by modules, routines, subroutines, or subparts of a computer program product.

If the specification states a component or feature “may”, “can”, “could”, or “might” be included or have a characteristic, that particular component or feature is not required to be included or have the characteristic.

Retinal diseases and disorders pose significant challenges to the healthcare industry, demanding precise and timely diagnostic solutions. Optical Coherence Tomography (OCT) has emerged as a powerful imaging technology for capturing detailed cross-sectional views of the retina, aiding in the diagnosis of conditions such as age-related macular degeneration, diabetic retinopathy, and glaucoma. However, the efficient and accurate classification of retinal OCT scans remains a complex task.

At its core, the ROI-Aware 2D ResNet represents a substantial departure from conventional methods. It places a central focus on the concept of the Region of Interest (ROI) within every scan. By doing so, it orchestrates a transformative shift in the diagnostic process. This unique approach significantly curtails computational overhead, leading to faster and more efficient results delivery.

ResNet-18, short for Residual Network 18, is a widely recognized and influential deep learning architecture. It is specifically designed for image classification tasks and belongs to the ResNet family of neural networks. ResNet-18 is celebrated for its exceptional performance and efficiency, offering a groundbreaking solution to the challenges of training very deep neural networks.

For experimentation purpose, a dataset is prepared. The dataset is structured into three main folders: “train,” “test,” and “val,” each containing subfolders corresponding to different image categories. These categories include “NORMAL,” “CNV” (Choroidal Neovascularization), “DME” (Diabetic Macular Edema), and “DRUSEN.” In total, the dataset comprises a substantial collection of 84,495 X-Ray images in JPEG format, classified into these four distinct categories.

To facilitate efficient organization, the images are distributed across four separate directories: “CNV” for Choroidal Neovascularization, “DME” for Diabetic Macular Edema, “DRUSEN” for drusen-related conditions, and “NORMAL” for healthy, non-pathological retinal scans. This meticulous organization allows for precise categorization and retrieval of retinal OCT scans for analysis and model training.

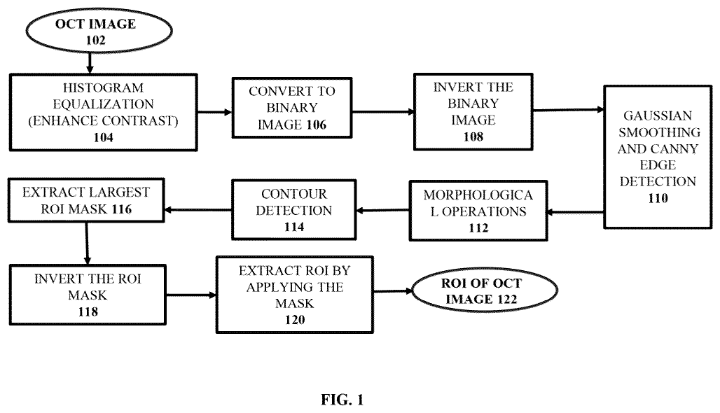

FIG. 1 illustrates brief steps for region of interest (ROI) extraction, in accordance with an exemplary embodiment of the present disclosure. A shown in FIG. 1, following are the essential key steps involved in ROI extraction as per the present invention. FIG. 1 shown following essential steps and its sequence to be followed for extraction.

As shown in FIG. 1, the system in accordance with the embodiments of the present invention, can include an image enhancer module 104, a converter module 106, an inverter module 108, an edge detection module 110 (performing gaussian smoothing and canny edge detection), a morphological operations module 112, a contour detection module 114, a largest ROI mask extraction module 116, an ROI mask inversion module 118 and an ROI extraction module 120, amongst other modules.

In an exemplary embodiment, the OCT images (102) can be obtained by scanning eye of a patient that generate an image (retinal scan). The patient may be at a remote location and plurality of such scans may be passed using means known in the art (using wi-fi and Internet, for example), to further components of the system that may be configured at a central location as needed.

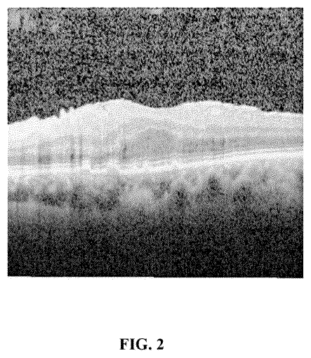

Enhancement of the image: This function employs a Histogram Equalization technique to adjust the pixel intensities in the image. By redistributing the pixel values, it enhances the overall contrast, making subtle details more pronounced. This step is essential for ensuring that the image is well-prepared for subsequent analysis, especially in scenarios with varying illumination conditions. FIG. 2 illustrates an image obtained upon performing image enhancement through histogram equalization, improving contrast, and revealing hidden details, as one of the ROI extraction steps, in accordance with an exemplary embodiment of the present disclosure.

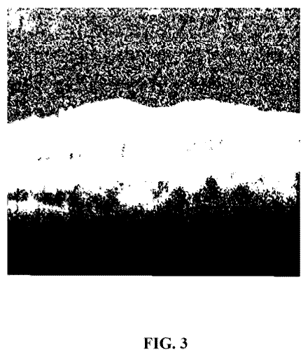

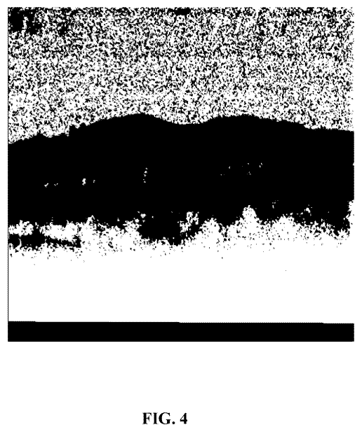

Conversion of binary image and inverted binary image: This process converts the enhanced image into a binary representation. Thresholding is applied to segment the image into two distinct regions: one representing important features (foreground) and the other for the less relevant portions (background). The inverted binary image provides a clear separation between these regions, simplifying further analysis. FIG. 3 illustrates an image obtained upon performing binary conversion: enhanced image transformed into a binary representation for further analysis, as one of the ROI extraction steps, in accordance with an exemplary embodiment of the present disclosure. FIG. 4 illustrates an image obtained upon performing inverted binary image, as one of the ROI extraction steps, in accordance with an exemplary embodiment of the present disclosure. This transformation reverses the values of a binary image, creating a negative rendition of the critical areas.

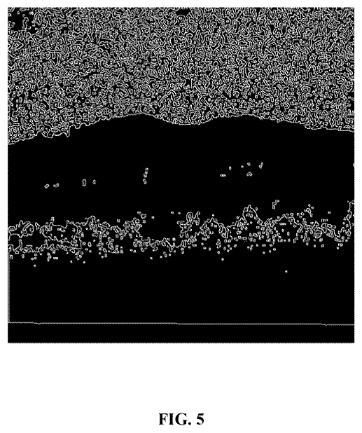

Perform edge detection: Utilizing Gaussian Blurring and Canny Edge Detection techniques, this step identifies edges and structural boundaries within the image. It identifies abrupt changes in pixel intensity, essentially tracing the contours of objects and features. The result is an image highlighting the structural aspects of the subject. FIG. 5 illustrates an image obtained upon performing canny edge detection, as one of the ROI extraction steps, in accordance with an exemplary embodiment of the present disclosure. The Canny edge detection algorithm is then employed to identify edges and transitions within the image

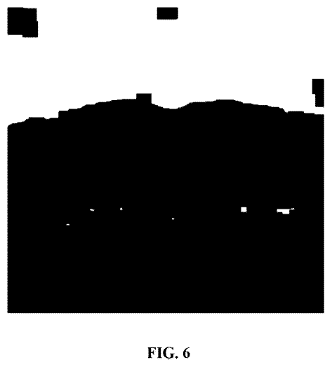

Morphological operations: Morphological operations like dilation and erosion refine the edges obtained from the previous step. Dilation expands prominent features, while erosion reduces noise and fine details. This process ensures that the image maintains well-defined structural information while minimizing unwanted artifacts. FIG. 6 illustrates an image obtained upon performing morphological operations, as one of the ROI extraction steps, in accordance with an exemplary embodiment of the present disclosure. The image is subjected to morphological operations, including dilation and erosion, to enhance specific features and remove noise

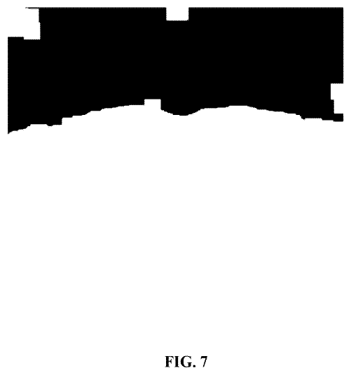

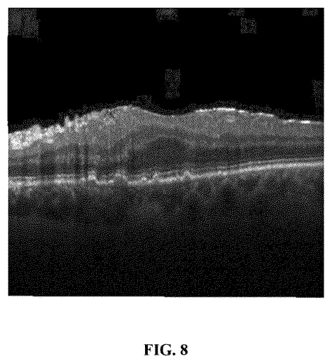

Find contours: This function locates contours within the image. It identifies continuous curves representing the boundaries of objects or structures. These contours play a critical role in defining regions of interest, enabling precise analysis and characterization. FIG. 7 illustrates an image obtained upon performing ROI mask, as one of the ROI extraction steps, in accordance with an exemplary embodiment of the present disclosure. This mask defines the Region of Interest (ROI), non-essential areas. FIG. 8 illustrates an image obtained upon performing cleared region removed from ROI, as one of the ROI extraction steps, in accordance with an exemplary embodiment of the present disclosure. Illustrating the removal of undesired areas from the Region of Interest (ROI).

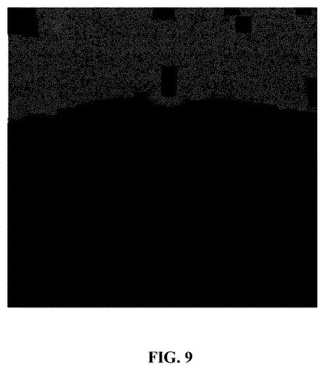

Extract largest ROI: Given a collection of identified contours, this step selects the most significant region of interest (ROI). It isolates the largest and most prominent area within the image, which is often the primary focus of the analysis. This ensures that the most relevant information is retained. FIG. 9 illustrates an image obtained upon performing difference image, as one of the ROI extraction steps, in accordance with an exemplary embodiment of the present disclosure. This image captures the contrast between the extracted Region of Interest (ROI) and the surrounding unwanted portions.

It may be appreciated that, inverting the ROI effectively emphasizes the background, providing valuable insight into the relationships between the key features and their surroundings. It serves as a visual representation of the innovative method's ability to isolate and eliminate non-critical areas, focusing solely on the essential elements for accurate retinal OCT classification.

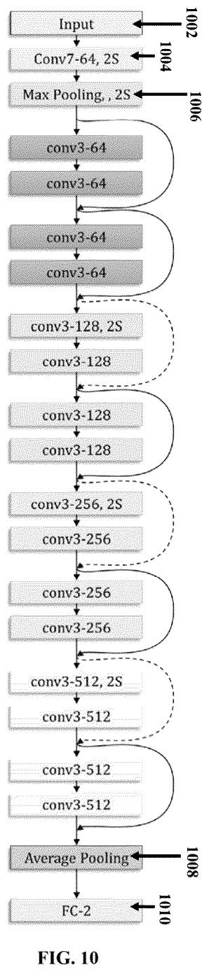

FIG. 10 illustrates an exemplary architecture of ResNet-18 (2D ResNet/3D ResNet) as well know in the art. For example in https://doi.org/10.1371/journal.pone.0256630.g005.

In an exemplary implementation, the ResNet-18 (2D ResNet/3D ResNet) may have following layers and respective processing at each layers:

Input Layer (1002): The model takes images as input, and the expected shape for the input is (224, 224, 3), where 224×224 is the image size, and 3 represents the three-color channels (RGB).

Initial Convolution Layer (1004): The first layer is a 2D convolutional layer with 64 filters, a kernel size of 7×7, and a stride of 2. This layer is responsible for capturing basic features from the input images. Batch normalization and ReLU activation functions are applied after this layer.

Max Pooling Layer (1006): After the initial convolution, there's a max-pooling layer with a pool size of 3×3 and a stride of 2. This reduces the spatial dimensions of the feature maps.

Residual Blocks: The core of the ResNet architecture is the residual blocks. The model defines three residual blocks. Each block consists of two convolutional layers with a specified number of filters and kernel size. The first convolution in each block is followed by batch normalization and ReLU activation. These blocks help the network learn more complex features and address the vanishing gradient problem.

Strided Convolutions: Some of the residual blocks use strided convolutions (stride=2) to reduce the spatial dimensions of the feature maps. This is a way to downsample the feature maps.

Global Average Pooling Layer (1008): After the last residual block, a global average pooling layer is applied. It computes the average of each feature map, resulting in a one-dimensional vector for each feature map. This reduces the spatial dimensions to 1×1, and it's a common way to convert the 2D feature maps into a flat vector for classification.

Output Layer (1010): The model ends with a dense (fully connected) layer with the number of units equal to the number of classes (num_classes). This layer applies the softmax activation function, which converts the raw scores into class probabilities.

Model Summary: The code defines the complete model using the Keras Functional API and prints a summary of the model's architecture, showing the layer types, output shapes, and the number of parameters in each layer.

The image classification process using the ResNet-18 architecture involves taking an input image, preprocessing it, and passing it through the model. ResNet-18's deep layers enable it to extract intricate features and patterns from the image. The model then assigns a class label to the image, providing a probability distribution over the possible categories. In the context of retinal OCT image classification, this means it can identify conditions like “NORMAL,” “CNV,” “DME,” and “DRUSEN.” The model's predictions can significantly assist healthcare professionals in swiftly and accurately diagnosing eye conditions based on retinal scans, ultimately improving patient care.

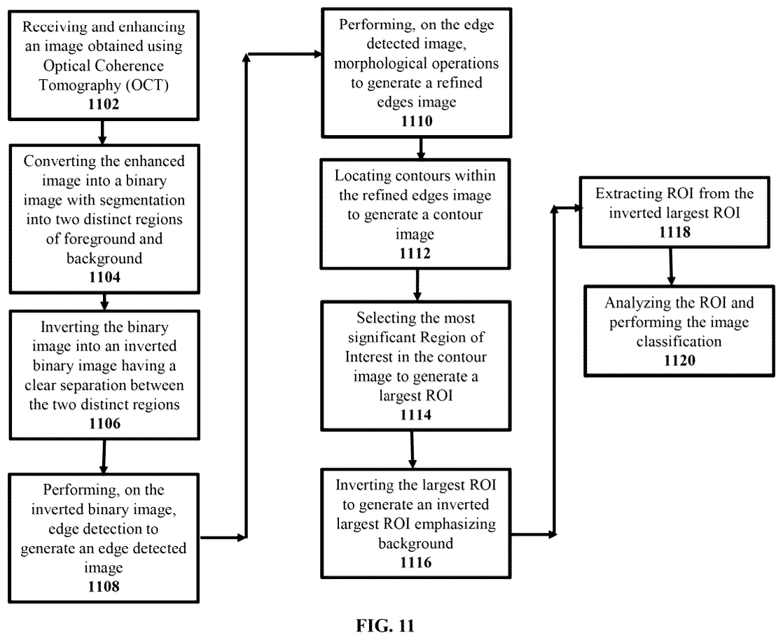

FIG. 11 elaborates upon a computer implemented method of performing classification of an image obtained using Optical Coherence Tomography (OCT), in accordance with an exemplary embodiment of the present disclosure.

As shown the method can include at step 1, receiving and enhancing an image obtained using Optical Coherence Tomography (OCT), as shown at 1102.

At step 2, the method can include converting the enhanced image into a binary image with segmentation into two distinct regions of foreground and background, as shown at 1104.

At step 3, the method can include inverting the binary image into an inverted binary image having a clear separation between the two distinct regions, as shown at 1106.

At step 4, the method can include performing, on the inverted binary image, edge detection to generate an edge detected image, as shown at 1108.

At step 5, the method can include performing, on the edge detected image, morphological operations to generate a refined edges image, as shown at 1110.

At step 6, the method can include locating contours within the refined edges image to generate a contour image, as shown at 1112.

At step 7 the method can include selecting the most significant Region of Interest (ROI) in the contour image to generate a largest ROI, as shown at 1114.

At step 8, the method can include inverting the largest ROI to generate an inverted largest ROI emphasizing background, as shown at 1116.

At step 9, the method can include extracting ROI from the inverted largest ROI, as shown at 1118; and

At step 10, the method can include analysing the ROI and performing the image classification as shown at 1120.

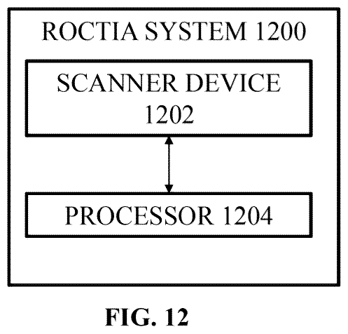

FIG. 12 relates to a preferred embodiment of the present invention which shows a retinal optical coherence tomography (OCT) image analysis (ROCTIA) system (1200) for analyzing one or more retinal scan images of an eye of a user to identify one or more retinal conditions of the eye, in accordance with an exemplary embodiment of the present disclosure.

The system includes a scanner device (1202) and a processor (1204) configured with a Region-of-Interest Aware (ROI-Aware) Residual Network (ResNet).

The scanner device (1202) configured to scan the eye of the user to obtain the one or more retinal scan images of the eye of the user.

The processor (1204) classifies each of the one or more retinal scan images based on a region of interest (ROI) in each of the one or more retinal scan images. The ROI is obtained in real-time while the one or more retinal scan images are obtained.

The processor (1204) identifies one or more retinal conditions of the eye based on at least one of the one or more retinal scan images.

In an exemplary embodiment, the ResNet is a ResNet-18 or a 2D ResNet or a 3D ResNet. In an implementation, the 2D ResNet is configured to operate on 2D images considering features associated with height and width dimensions, and the 3D ResNet is configured to operate on 3D images considering features associated with depth, height, and width dimensions.

In an exemplary embodiment, to obtain the ROI, the processor is configured to enhance the one or more retinal scan images obtained from the scanner; convert of the one or more retinal scan images into one or more binary representation; identify edges and structural boundaries within the one or more retinal scan images by utilizing gaussian blurring and canny edge detection techniques to obtain the one or more images highlighting structural aspects of the user; determine one or more contours within the one or more highlighted images; and obtain at least one contour from the one or more contours indicative of the ROI. The at least one contour is selected based on an area within at least one image from the one or more highlighted images.

In an exemplary embodiment, the processor is configured to perform dilation and erosion to refine the edges and structural boundaries within the one or more retinal scan images.

In an exemplary embodiment, the one or more retinal conditions is selected from diabetic retinopathy, glaucoma, age macular degeneration, and detached retina.

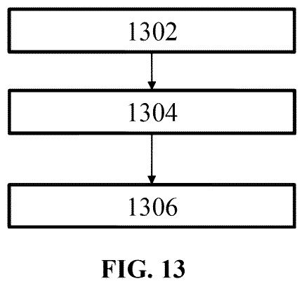

FIG. 13 relates to a method for analyzing one or more retinal scan images of an eye of a user to identify one or more retinal conditions of the eye, in accordance with an exemplary embodiment of the present disclosure.

At step 1302, a scanner device scans the eye of the user to obtain the one or more retinal scan images of the eye of the user.

At step 1304, a processor configured with a Region-of-Interest Aware (ROI-Aware) Residual Network (ResNet) classifies each of the one or more retinal scan images based on a region of interest (ROI) in each of the one or more retinal scan images. The ROI is obtained in real-time while the one or more retinal scan images are obtained.

At step 1306, the processor identifies one or more retinal conditions of the eye based on at least one of the one or more retinal scan images.

In an exemplary embodiment, the ResNet is a ResNet-18 or a 2D ResNet or a 3D ResNet. In an implementation, the 2D ResNet is configured to operate on 2D images considering features associated with height and width dimensions, and the 3D ResNet is configured to operate on 3D images considering features associated with depth, height, and width dimensions.

In an exemplary embodiment, to obtain the ROI, the processor is configured to enhance the one or more retinal scan images obtained from the scanner; convert of the one or more retinal scan images into one or more binary representation; identify edges and structural boundaries within the one or more retinal scan images by utilizing gaussian blurring and canny edge detection techniques to obtain the one or more images highlighting structural aspects of the user; determine one or more contours within the one or more highlighted images; and obtain at least one contour from the one or more contours indicative of the ROI. The at least one contour is selected based on an area within at least one image from the one or more highlighted images.

In an exemplary embodiment, the processor is configured to perform dilation and erosion to refine the edges and structural boundaries within the one or more retinal scan images.

In an exemplary embodiment, the one or more retinal conditions is selected from diabetic retinopathy, glaucoma, age macular degeneration, and detached retina.

In an exemplary embodiment, the one or more retinal scan images are classified using ResNet-18 architecture that receives the one or more retinal scan images as an input image, preprocess it, and pass it through the ResNet-18 architecture. The one or more deep layers extracts intricate features and patterns from the input image, and then assigns a class label to the input image, providing a probability distribution over the possible categories.

Various components such as modules as above and further described can be configured using hardware and software and various algorithms. These components can be configured to be in communication with one another and transfer data to one another as needed, using means and techniques known. Further, as can be readily understood any of these components/modules may be combined as needed, or, equally, be split into further components/modules as needed.

At its core, the ROI-Aware 2D ResNet represents a substantial departure from conventional methods. It places a central focus on the concept of the Region of Interest (ROI) within every scan. By doing so, it orchestrates a transformative shift in the diagnostic process. This unique approach significantly curtails computational overhead, leading to faster and more efficient results delivery.

As discussed earlier, retinal diseases and disorders present formidable challenges in the realm of healthcare diagnostics, necessitating precise and swift diagnostic methodologies. Optical Coherence Tomography (OCT) has emerged as a pivotal imaging technology for scrutinizing detailed cross-sectional views of the retina, aiding in the identification of conditions such as age-related macular degeneration, diabetic retinopathy, and glaucoma. Despite the advancements in OCT imaging, the accurate classification of retinal scans remains intricate.

The present invention, referred to as the ROI-Aware 2D ResNet, represents a paradigm shift from conventional methodologies, placing a focal emphasis on the Region of Interest (ROI) within each scan. This distinctive approach not only transforms the diagnostic process but also significantly reduces computational overhead, ensuring expeditious and efficient delivery of results.

The invention disclosed has the following technical advancements as compared to the conventional technologies. The ROI-Aware 2D ResNet has several comparative advantages in contrast to prior art, including established deep learning architectures such as ResNet-18.

Artificial intelligence registration and marker detection, including machine learning and using results thereof: The key distinction lies in the specific medical imaging focus and methodology. The ROI-Aware 2D ResNet for retinal OCT prioritizes precise classification of retinal conditions using ResNet-18 and a specialized ROI extraction process. In contrast, the conventional technologies spans various imaging modalities, employing artificial intelligence for tasks like registration, marker detection, and diverse machine learning models. This reveals a targeted approach for retinal OCT versus a more generalized strategy applicable to multiple imaging scenarios.

The use of GUI interactions (not explicitly disclosed by the system can have a GUI) in present disclosure pertains to selecting the region of interest (ROI) and positioning markers, angles, or planes during procedures like angiography and intravascular imaging, particularly in Percutaneous Coronary Intervention (PCI). While both applications involve defining an ROI, the key difference lies in the nature of the imaging. In angiography, real-time user interactions are crucial for guiding catheters and gaining precise information about vessels, lumen size, and plaque morphology. In contrast, the ROI-Aware 2D ResNet for retinal OCT classification focuses on automated extraction of ROIs from static retinal OCT scans, where user-driven real-time adjustments are not applicable. The GUI interactions in angiography serve an interventional purpose, distinct from the automated and non-invasive nature of retinal OCT classification.

It's important to note that the use of ResNet architecture is a common practice in deep learning models. However, in contrast, the ROI-Aware 2D ResNet for retinal OCT classification employs ResNet-18, a specific variant designed for image classification tasks. The focus on the Region of Interest (ROI) within retinal scans is a distinctive aspect of the invention elaborated herein, streamlining the diagnostic process and reducing computational overhead. While both architectures may share a foundation in ResNet, the specific adaptations and purposes differ, with the invention disclosed focused specifically upon the challenges of retinal OCT classification.

It may be appreciated from the above disclosure that the Region-of-Interest (ROI) aware 2D ResNet represents a specialized neural network architecture with a specific focus on image classification tasks, particularly within the designated Region of Interest. The primary distinction, when compared to the conventional technologies, lies in the comprehensive versatility of the acquisition devices in contrast to the targeted nature of ROI aware 2D ResNet, designed for precise analysis within specific medical imaging contexts.

It may be appreciated from the above disclosure that, the distinction between slice determination (as used in conventional technology for retina image analysis) and extracting the Region of Interest (ROI) from Optical Coherence Tomography (OCT) images lies in their fundamental objectives and methodologies. Slice determination, as described in the provided text, revolves around the classification of specific slices within medical images, focusing on anatomical regions such as the head, neck, and chest. This process entails training a slice classification model, leveraging a 2D ResNet network structure and input from an imaging doctor who labels key slices for gold standard classification.

On the other hand, extracting ROI from OCT images targets the precise identification and isolation of relevant regions within OCT scans, particularly in the context of retinal diseases. The emphasis here is on pinpointing the region of interest for diagnostic or analytical purposes, potentially involving specific adaptations in network architecture or labeling techniques tailored to the characteristics of OCT images.

It may be appreciated from the above disclosure that the enhanced Optical Coherence Tomography (EOCT) model is centered around retinal OCT image classification, employing a modified ResNet pretrained architecture and the random forest algorithm with dual SGD and Adam optimizers. While aiming for improved performance on retinal images, the EOCT model does not explicitly emphasize region-of-interest awareness. In contrast, the previously discussed ROI-Aware Retinal OCT model is specifically designed to classify retinal OCT images with a distinct focus on the Region of Interest. Utilizing a 2D ResNet architecture, it aims to extract and analyse relevant regions for accurate image classification. The ROI-Aware Retinal OCT model differentiates itself through its explicit consideration of the region of interest within the retinal scans, providing a specialized approach to image analysis.

Specific Characteristics Differences of OCT (as Used in the Present Invention) and CT/MRI (as Conventional Technology):

Depth and Resolution: OCT excels in capturing high-resolution images with remarkable clarity, particularly in superficial biological tissues such as the retina. The micrometer-scale resolution of OCT allows for detailed examination of fine structures, making it an invaluable tool in ophthalmology for visualizing intricate layers of the retina. On the other hand, CT and MRI, while offering excellent imaging depth for visualizing deeper anatomical structures, may not match the level of resolution achieved by OCT in capturing surface details.

Principles of imaging: OCT relies on low-coherence interferometry, employing interference patterns of light to create high-resolution cross-sectional images, particularly effective for imaging thin biological tissues like the retina. In contrast, CT relies on X-ray attenuation to produce detailed cross-sectional images of internal structures, while MRI utilizes nuclear magnetic resonance principles to generate anatomical images, excelling in soft tissue imaging.

Application Specifics: OCT is particularly tailored for applications in ophthalmology, offering unparalleled insights into retinal structures and pathologies. Its high-resolution imaging capabilities make it an invaluable tool for diagnosing and monitoring conditions such as diabetic retinopathy, macular edema, and age-related macular degeneration. CT, utilizing X-rays, is particularly adept at swiftly producing high-resolution cross-sectional images, making it invaluable for diagnosing a spectrum of conditions such as fractures, tumors, and vascular diseases, especially in dense structures like bones. On the other hand, Magnetic Resonance Imaging employs magnetic fields and radiofrequency pulses, excelling in soft tissue imaging for neurological studies, musculoskeletal assessments, abdominal and pelvic imaging, and breast examinations.

Speed of imaging: OCT is renowned for its rapid imaging capabilities, offering real-time visualization of high-resolution cross-sectional images. This swift imaging is particularly advantageous in ophthalmology, allowing dynamic examination of retinal structures with minimal motion artifacts. In contrast, CT scans are relatively quick, taking seconds for image acquisition, but may involve additional time for processing. MRI, known for detailed soft tissue imaging, generally has longer acquisition times, ranging from minutes to over an hour. While CT and MRI provide comprehensive anatomical insights, the rapid imaging of OCT proves crucial in situations requiring immediate assessments or dynamic monitoring.

Non-invasiveness: The non-invasiveness of Optical Coherence Tomography (OCT) stands as a key distinction from Computed Tomography (CT) and Magnetic Resonance Imaging (MRI). OCT's utilization of light waves for imaging renders it inherently non-invasive, particularly advantageous in ophthalmology for examining retinal structures without the need for surgical interventions or injections. This non-invasive approach not only ensures patient comfort but also contributes to the safety and accessibility of the imaging process. In contrast, both CT and MRI, while invaluable in medical diagnostics, may involve invasive elements such as the administration of contrast agents through injection, highlighting the unique advantage of OCT in providing detailed imaging with minimal impact on the patient.

Processing Optical Coherence Tomography (OCT) images with a 2D ResNet and Magnetic Resonance Imaging (MRI)/Computed Tomography (CT) images with a ResNet involves several key differences owing to the unique characteristics of the respective imaging modalities

Architectural Disparities: The architectural disparities between the 2D ResNet designed for OCT images and the 3D ResNet tailored for CT/MRI scans are rooted in their respective approaches to spatial information processing. The 2D ResNet operates on 2D images, addressing features in the height and width dimensions. It employs 2D convolutions for efficient feature extraction within this two-dimensional space, resulting in a computationally lighter model with fewer parameters. The depth of the network may not need to be as extensive as the 3D ResNet, given the simpler spatial context.

Conversely, the 3D ResNet is engineered for 3D volumetric data, necessitating the consideration of features across depth, height, and width dimensions. It deploys 3D convolutions to capture spatial dependencies in three dimensions, enabling the modeling of volumetric structures. This approach introduces a larger number of parameters due to the heightened spatial complexity, demanding more computational resources. The depth of the 3D ResNet often needs to be deeper to effectively capture intricate spatial relationships within volumetric data.

In practical terms, the choice between these architectures involves trade-offs. The 2D ResNet proves suitable for planar images like OCT scans, offering computational efficiency. Meanwhile, the 3D ResNet becomes indispensable for volumetric data in CT/MRI, albeit at the cost of increased computational demands and model complexity. Careful consideration of these factors is crucial in selecting the most apt model for a specific imaging context.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices are able to exchange data with each other over the network, possibly via one or more intermediary device.

Hence while embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

To reiterate, while the invention has been explained with reference to the specific embodiment of the invention, the explanation is illustrative and the invention is limited only by the appended claims.

### Advantages of the Invention

Present disclosure provides for a system to advance the field of ophthalmic healthcare by automating and enhancing the analysis of retinal OCT scans.

Present disclosure provides for a system that provides consistently accurate and reliable diagnoses by automating the identification of relevant regions within OCT scans.

Present disclosure provides for a system to expedite the diagnostic process, enabling timely treatment for eye conditions and reducing the workload on medical professionals.

Present disclosure provides for a system that makes expert-level retinal OCT analysis more accessible, bridging the gap in regions with limited ophthalmic expertise.

Present disclosure provides for a system that is scalable and can address the increasing volume of retinal OCT scans with a systematic and efficient approach, ensuring high-quality eye care services

We claim:

1. A retinal optical coherence tomography (OCT) image analysis (ROCTIA) system (1200) for analyzing one or more retinal scan images of an eye of a user to identify one or more retinal conditions of the eye, the ROCTIA system comprising:
a scanner device (1202) configured to scan the eye of the user to obtain the one or more retinal scan images of the eye of the user;
a processor (1204) configured with a Region-of-Interest Aware (ROI-Aware) Residual Network (ResNet), that enables the processor to:
classify each of the one or more retinal scan images based on a region of interest (ROI) in each of the one or more retinal scan images, wherein the ROI is obtained in real-time while the one or more retinal scan images are obtained; and
identify one or more retinal conditions of the eye based on at least one of the one or more retinal scan images.

2. The ROCTIA system as claimed in claim 1, wherein the ResNet is a ResNet-18 or a 2D ResNet or a 3D ResNet, and wherein the 2D ResNet is configured to operate on 2D images considering features associated with height and width dimensions, and the 3D ResNet is configured to operate on 3D images considering features associated with depth, height, and width dimensions.

3. The ROCTIA system as claimed in claim 1, wherein to obtain the ROI, the processor is configured to:
enhance the one or more retinal scan images obtained from the scanner;
convert of the one or more retinal scan images into one or more binary representation;
identify, by utilizing gaussian blurring and canny edge detection techniques, edges and structural boundaries within the one or more retinal scan images to obtain the one or more images highlighting structural aspects of the user;
determine one or more contours within the one or more highlighted images;
obtain at least one contour from the one or more contours indicative of the ROI, wherein the at least one contour is selected based on an area within at least one image from the one or more highlighted images.

4. The ROCTIA system as claimed in claim 3, wherein the processor is configured to:
perform dilation and erosion to refine the edges and structural boundaries within the one or more retinal scan images.

5. The ROCTIA system as claimed in claim 1, wherein the one or more retinal conditions is selected from diabetic retinopathy, glaucoma, age macular degeneration, and detached retina.

6. A method for analyzing one or more retinal scan images of an eye of a user to identify one or more retinal conditions of the eye, the method being implemented by retinal optical coherence tomography (OCT) image analysis (ROCTIA) system, the method comprising:
scanning (1302), by a scanner device, the eye of the user to obtain the one or more retinal scan images of the eye of the user;
classifying (1304), by a processor configured with a Region-of-Interest Aware (ROI-Aware) Residual Network (ResNet), each of the one or more retinal scan images based on a region of interest (ROI) in each of the one or more retinal scan images, wherein the ROI is obtained in real-time while the one or more retinal scan images are obtained; and
identifying (1306), by the processor, one or more retinal conditions of the eye based on at least one of the one or more retinal scan images.

7. The method as claimed in claim 6, wherein the step of obtaining the ROI includes:
enhancing, by the processor, the one or more retinal scan images obtained from the scanner;
converting, by the processor, of the one or more retinal scan images into one or more binary representation;
identifying, by the processor, by utilizing gaussian blurring and canny edge detection techniques, edges and structural boundaries within the one or more retinal scan images to obtain the one or more images highlighting structural aspects of the user;
performing, by the processor, dilation and erosion to refine the edges and structural boundaries within the one or more retinal scan images;
determining, by the processor, one or more contours within the one or more highlighted images;
obtaining, by the processor, at least one contour from the one or more contours indicative of the ROI, wherein the at least one contour is selected based on an area within at least one image from the one or more highlighted images.

8. The method as claimed in claim 6, wherein the ResNet is a ResNet-18 or a 2D ResNet or a 3D ResNet, and wherein the 2D ResNet is configured to operate on 2D images considering features associated with height and width dimensions, and the 3D ResNet is configured to operate on 3D images considering features associated with depth, height, and width dimensions.

9. The method as claimed in claim 6, wherein the one or more retinal conditions is selected from diabetic retinopathy, glaucoma, age macular degeneration, and detached retina.

10. The method as claimed in claim 6, wherein the one or more retinal scan images are classified using ResNet-18 architecture that receives the one or more retinal scan images as an input image, preprocess it, and pass it through the ResNet-18 architecture, wherein one or more deep layers extracts intricate features and patterns from the input image, and then assigns a class label to the input image, providing a probability distribution over the possible categories.
